(19) United States
(12) Patent Application Publication
(10) Pub. No.: US 2016/0205943 A1
(43) Pub. Date: Jul. 21, 2016

(54) Technique, Method, and Composition for Controlling Plant Pathogens

(71) Applicant: KOREA BIO CHEMICAL CO., LTD., Jinju-si (KR)

(72) Inventors: Hoon Serg KANG, Changwon-si (KR); Jae Gon KANG, Jinju-si (KR); Sang Hoon PAIK, Jinju-si (KR); Jeong Chan PARK, Jinju-si (KR); Sang Hoon HAN, Changwon-si (KR); Chang Seuk PARK, Jinju-si (KR); Young Ui LEE, Jinju-si (KR)

(73) Assignee: Korea Bio Chemical Co., Ltd., Jinju-si (KR)

(21) Appl. No.: 14/784,564

(22) PCT Filed: Feb. 21, 2013
(86) PCT No.: PCT/KR2013/001375

§ 371 Date: Oct. 14, 2015

(30) Foreign Application Priority Data
Feb. 21, 2013 (KR) 10-2013-0018512

(51) Int. Cl. A01N 63/02; C12N 1/20; C12P 1/04; C12R 1/07

(52) U.S. Cl. CPC A01N 63/02; C12R 1/07; C12N 1/20; C12P 1/04

(57) ABSTRACT

The present invention relates to a technique, a method, and a composition for controlling plant pathogens comprising a Bacillus amyloliquefaciens KBC1004 strain and a culture medium thereof as active ingredients. Particularly, the Bacillus amyloliquefaciens KBC1004 strain and the culture medium thereof of the present invention have excellent antifungal activity against various plant pathogens such as Rhizoctonia solani AG-2-2(IV) causing turfgrass diseases, Botrytis cinerea causing gray mold rot, Colletotrichum acutatum causing chili pepper anthracnose, Colletotrichum gleosprodes causing sweet persimmon anthracnose, Rhizoctonia cerealis causing yellow patch, Rhizoctonia solani AG-1(1A) causing brown patch, Sclerotium rolfsii causing southern blight, and Phytophthora drechsleri causing kiwifruit phytophthora blight, so that they can be efficiently used as an eco-friendly composition for controlling plant pathogens.

### BACKGROUND OF THE INVENTION

1. Field of the Invention

The present invention relates to a technique, a method, and a composition for controlling plant pathogens comprising the Bacillus amyloliquefaciens KBC1004 strain and the culture medium thereof as active ingredients.

2. Description of the Related Art

Environmental & ecosystem conservation is the major matter of international concern. The overuse of organosynthetic agricultural chemicals for controlling the plant disease causes soli pollution, environmental/ecosystem destruction, and toxicity on animal, etc. Particularly, the consumer's concern about the pollution in agricultural ecosystem caused by the misuse/overuse of such organosynthetic agricultural chemicals turns to the interest in pro-environmental safe agricultural products. To eliminate the side effects of such organosynthetic agricultural chemicals, the biological control system acting against plant pathogens has been studied with microorganisms playing a role of controlling in the ecosystem and their metabolites. The biggest advantage of such natural plant protector is the minimum effect on environmental and agricultural ecosystem. However, since the natural plant protector is a living organism, the preservation period at room temperature is short. Most of the microorganisms industrialized as a natural plant protector belong to Bacillus sp., which is because those microorganisms are able to form endospores that endure environmental stresses such as heat and dry, etc. Bacillus is widely spread in the nature and produces a peptide like material that has multiple function which favors not only the antibiotic activity against plant pathogens and plant defense mechanism to enhance plant growth but also the colonization in rhizosphere.

The study on the natural plant protector began in the early 1970s, which was mainly lead by government research institutes, university research institutes, and companies. The most successful natural plant protectors are Galltro-A, Nogall/Diegall, and Norbac84C, which had been developed by using Agrobacterium radiobacter and are now registered products. In 2003, Agraquest Co., USA, developed Serenade, and this licensed product is now on world-wide markets of 25 countries by the distribution through the multinational agricultural company BASF. In Korea, about 30 kinds of natural plant protectors have been registered and used so far. In particular, species of microorganisms have been used as a germicide, among which 13 species are the members of Bacillus sp.

Bacillus sp. strain, which has been used as a biological control agent for plant disease, is a Gram positive, non-pathogenic microorganism having endospores and easy to culture. This strain is also known to produce various enzymes such as protease, amylase, glucanase, and cellulase, and antimicrobial compounds having different structures as well, suggesting that this microorganism is industrially important so that it is widely used as a host microorganism in biological industry.

In the meantime, as the composition for controlling plant pathogens using Bacillus amyloliquefaciens, KB3 strain (Korean Patent application No. 10-2011-0065439), CS61 strain (Korean Patent application No. 10-2011-0029387), KB-MJK 601 strain (Korean Patent application No. 10-2011-0014038), IN937a strain (Korean Patent application No. 10-2010-0133116), JBC36 strain (Korean Patent No. 1189104), EML-BS2 strain (Korean Patent application No. 10-2010-0065557), CP1 strain (Korean Patent application No. 10-2010-0040303), GIB-01 strain (Korean Patent application No. 10-2009-0037218), CC175 strain (Korean Patent No. 838103), A-7 strain (Korean Patent No. 868901), LP03 strain (Korean Patent No. 807403), KS-4 strain (Korean Patent No. 781472), KTGB0202 strain (Korean Patent No. 535912), LX9 strain (Korean Patent No. 472376), and KM112 strain (Korean Patent No. 4295586) have been reported to display anti-fungal activity.

Even though many microbial control agents showing anti-fungal activity against plant pathogens including the said Bacillus amyloliquefaciens strain have been reported, the effect of controlling various plant diseases is still not satisfactory, requiring a novel biological microbial control agent.

According to the prior art, the microorganism acting against pathogens has been used for biological controlling. However, the active concentration of the material showing anti-bacterial activity is so low that biological controlling effect decreases.

The present inventors tried to develop a novel strain and material having excellent antifungal activity against various plant pathogens. As a result, the inventors confirmed that the Bacillus amyloliquefaciens KBC1004 strain and the culture medium thereof had excellent antifungal activity against various pathogens such as Rhizoctonia solani AG-2-2(IV) causing turfgrass diseases, Botrytis cinerea causing gray mold rot, Colletotrichum acutatum causing chili pepper anthracnose, Colletotrichum gleosprodes causing sweet persimmon anthracnose, Rhizoctonia cerealis causing yellow patch, Rhizoctonia solani AG-1(1A) causing brown patch, Sclerotium rolfsii causing southern blight, and Phytophthora drechsleri causing kiwifruit phytophthora blight, and further established a technique and a method to shrink or kill the pathogens by exchanging specific sensitizing materials, leading to the completion of this invention.

### SUMMARY OF THE INVENTION

It is an object of the present invention to provide the novel Bacillus amyloliquefaciens KBC1004 strain.

It is another object of the present invention to provide a technique, a method, and a composition for controlling plant pathogens comprising the said strain or the culture medium thereof as an active ingredient.

To achieve the above objects, the present invention provides a novel Bacillus amyloliquefaciens KBC1004 strain deposited under the Accession No. of KCTC 12355BP.

The present invention also provides a technique, a method, and a composition for controlling plant pathogens comprising, as an active ingredient, the said strain, the culture medium, or the sensitizing material secreted by the interaction with the plant pathogens.

The present invention further provides a technique, a method, and a composition for controlling turfgrass rhizoctonia blight (large patch) caused by Rhizoctonia solani AG-2-2(IV) comprising, as an active ingredient, the said strain, the culture medium, or the sensitizing material secreted by the interaction with the plant pathogens.

In addition, the present invention provides a method for preparing a composition for controlling plant pathogens comprising the following steps:

1) preparing a culture product by culturing the Bacillus amyloliquefaciens KBC1004 strain deposited under the Accession No. of KCTC 12355BP in a liquid medium; and

2) preparing a raw material by drying the culture product prepared in step 1) and pulverizing thereof.

### Advantageous Effect

The Bacillus amyloliquefaciens KBC1004 strain and the culture medium of the present invention display excellent antifungal activity against various plant pathogens such as Rhizoctonia solani AG-2-2(IV) causing turfgrass diseases, Botrytis cinerea causing gray mold rot, Colletotrichum acutatum causing chili pepper anthracnose, Colletotrichum gleosprodes causing sweet persimmon anthracnose, Rhizoctonia cerealis causing yellow patch, Rhizoctonia solani AG-1(1A) causing brown patch, Sclerotium rolfsii causing southern blight, and Phytophthora drechsleri causing kiwifruit phytophthora blight, so that they can be efficiently used for the eco-friendly technique, method, and composition for controlling plant pathogens.

### BRIEF DESCRIPTION OF THE DRAWINGS

The application of the preferred embodiments of the present invention is best understood with reference to the accompanying drawings, wherein:

FIG. 1 is a diagram illustrating the 16s rDNA sequence of the novel Bacillus amyloliquefaciens KBC1004 strain of the present invention.

FIG. 2 is a diagram illustrating the identification of the novel Bacillus amyloliquefaciens KBC1004 strain of the present invention by ANI analysis.

FIG. 3 is a diagram illustrating the HPLC profile of the acid precipitate of Bacillus amyloliquefaciens KBC1004 strain culture medium.

FIG. 4 is a diagram illustrating the result of HPLC performed to confirm the antibacterial material produced by the Bacillus amyloliquefaciens KBC1004 strain.

FIG. 5a is a diagram illustrating the result of LC-mass performed to confirm the antibacterial material produced by the Bacillus amyloliquefaciens KBC1004 strain.

FIG. 5b is a diagram presenting the mass values of peak #1˜peak #3 of FIG. 5a.

FIG. 5c is a diagram presenting the mass values of peak #4˜peak #6 of FIG. 5a.

FIG. 5d is a diagram presenting the mass values of peak #7˜peak #9 of FIG. 5a.

FIG. 5e is a diagram presenting the mass values of peak #10˜peak #12 of FIG. 5a.

FIG. 6 is a diagram illustrating the replacement culture of Rhizoctonia solani AG-2-2(IV) with the Bacillus amyloliquefaciens KBC1004 strain extract, inhibition zone extract, culture concentrate, and acid precipitate.

FIG. 7 is a diagram illustrating the sensitizing material production by the replacement culture of Rhizoctonia solani AG-2-2(IV) and the Bacillus amyloliquefaciens KBC1004 strain.

### DESCRIPTION OF THE PREFERRED EMBODIMENTS

Hereinafter, the present invention is described in detail.

The present invention provides the novel Bacillus amyloliquefaciens KBC1004 strain deposited under the Accession No. of KCTC 12355BP.

The said strain preferably displays antibacterial activity against one or more plant pathogens selected from the group consisting of Rhizoctonia solani AG-2-2(IV), Botrytis cinerea, Colletotrichum acutatum, Colletotrichum gleosprodes, Rhizoctonia cerealis, Rhizoctonia solani AG-1(1A), Sclerotium rolfsii, and Phytophthora drechsleri, but not always limited thereto.

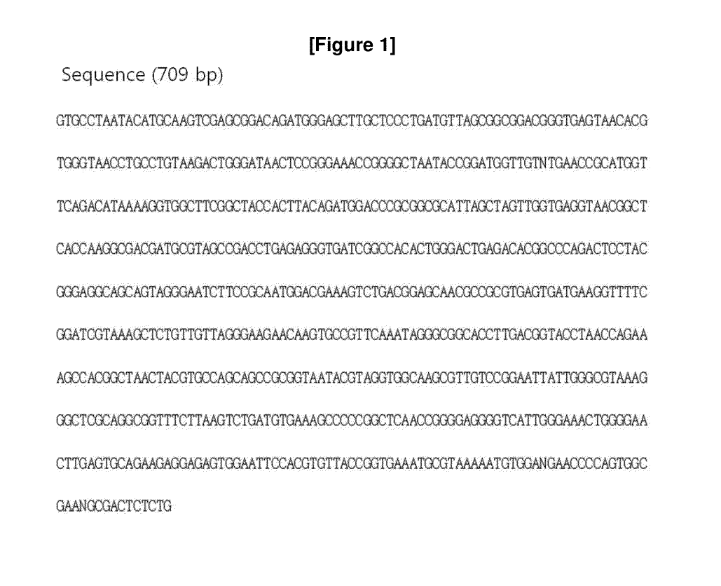

In a preferred embodiment of the present invention, the inventors selected a strain that showed excellent antibacterial activity among the microorganisms separated from the soil, and then identified its sequence (see SEQ. ID. NO: 1 and FIG. 1). Then, the inventors performed homology search by using NCBI GenBank database and confirmed its phylogenetic position. From the molecular phylogenetic analysis, the selected strain was identified as Bacillus amyloliquefaciens belonging to the Bacillus sp. phylogenetic group.

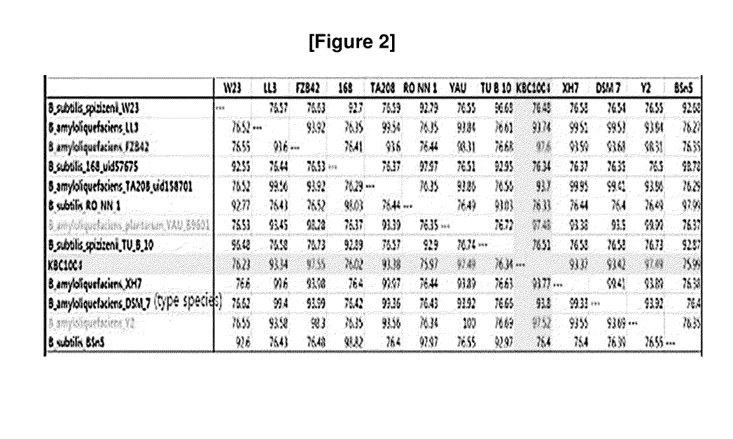

The selected strain was also identified as Bacillus amyloliquefaciens through genome analysis (see FIG. 2). It was confirmed by genome annotation that the strain contained the genome involved in the biosynthesis of surfactin, bacillomycin D-like antibiotics, fengycin, putative peptide, bacillibactin, bacilysin/anticapsin, macrolactin, bacillaene, and difficidin (see Table 1).

The separated and identified strain was named Bacillus amyloliquefaciens KBC1004 and deposited at Korean Collection for Type Cultures (KCTC), Korea Research Institute of Bioscience and Biotechnology (KRIBB) on Jan. 18, 2013 (Accession No: KCTC 12355BP).

The enzyme produced by the novel Bacillus amyloliquefaciens KBC1004 strain was investigated. As a result, it was confirmed that the novel strain of the present invention produced Esterase (C4) and Naphtol-AS-BI-phosphohydrolase (see Table 2). The carbohydrate usability of the strain was investigated and the results are presented in Table 3 (see Table 3).

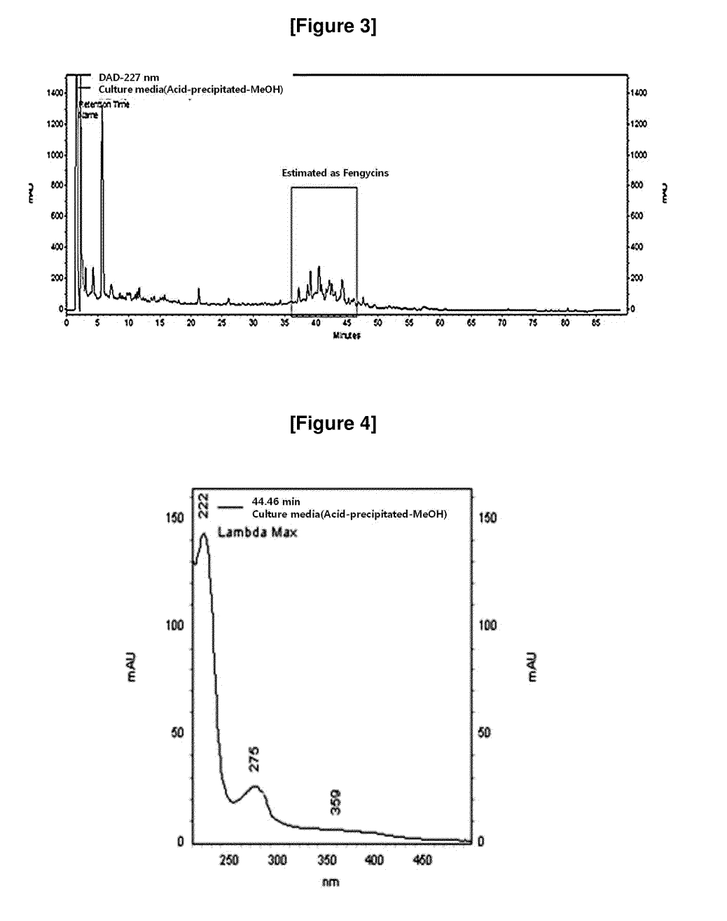

To investigate the antibiotics produced by the novel Bacillus amyloliquefaciens KBC1004 strain of the present invention, HPLC was performed with the strain culture medium acid precipitate. As a result, multiple peaks were detected in the retention time of 37˜45 minutes. Along with the analysis of the retention time of the peak, UV spectrum presenting the maximum absorbance at 222 and 275 nm also suggested that the antibiotic material produced by the strain was fengycin cluster, the lipopeptide antibiotic (see FIGS. 3 and 4).

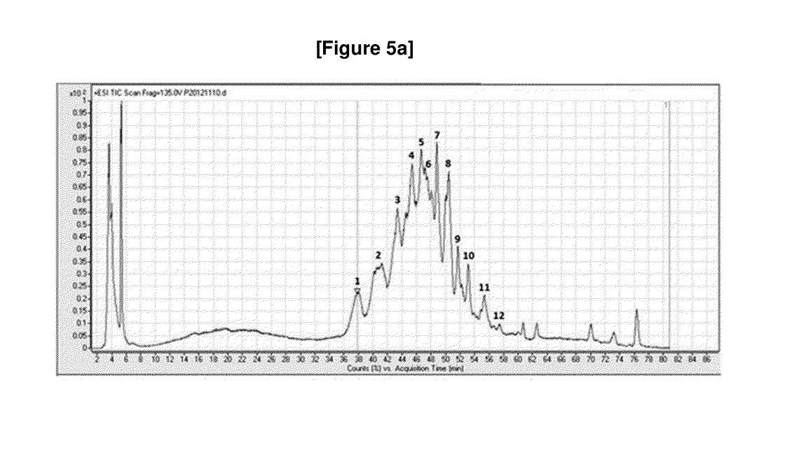

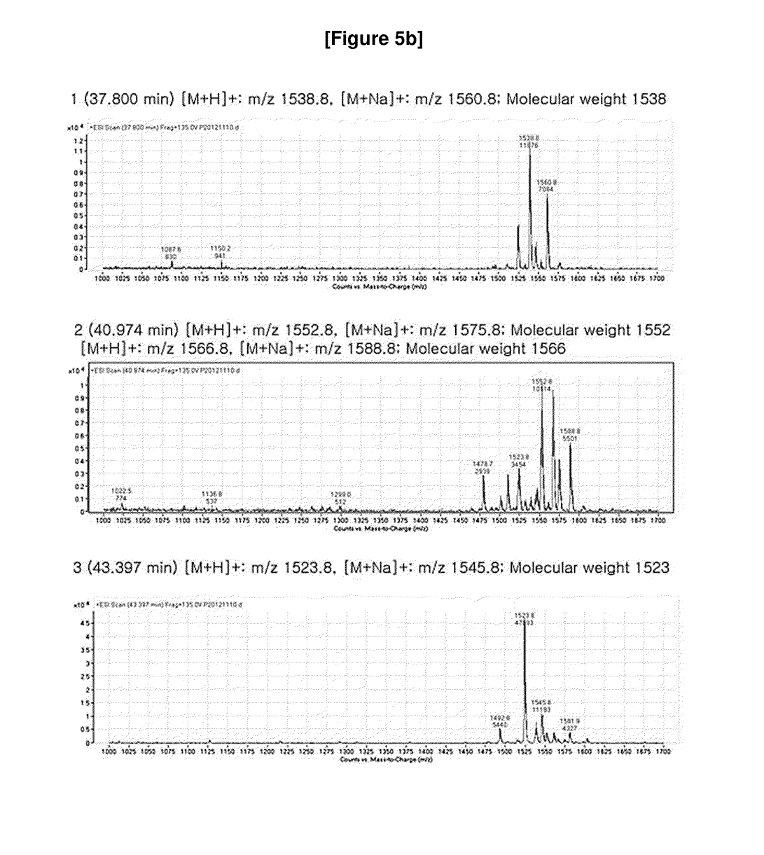

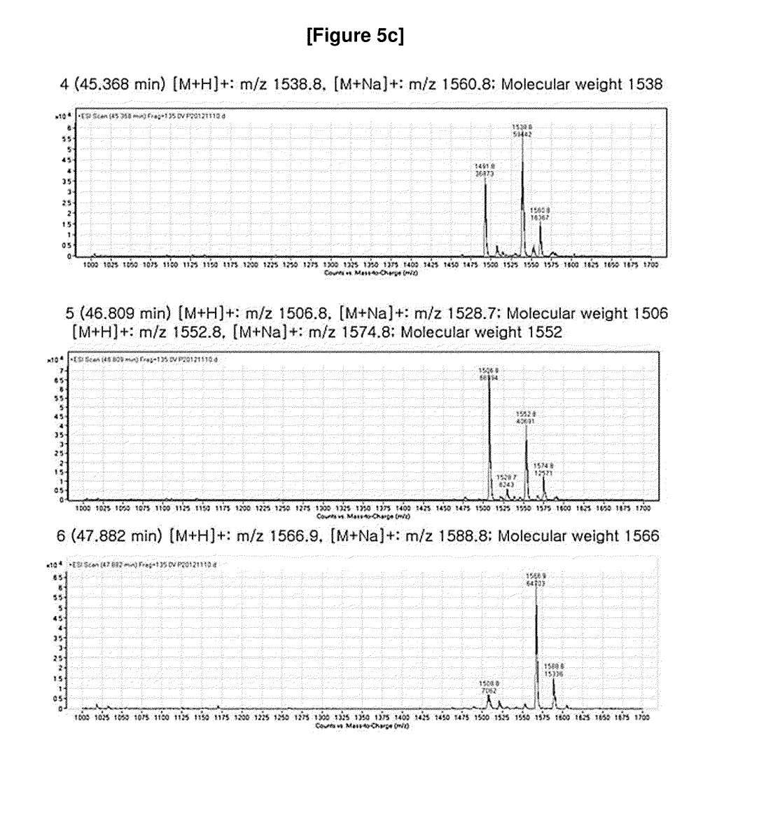

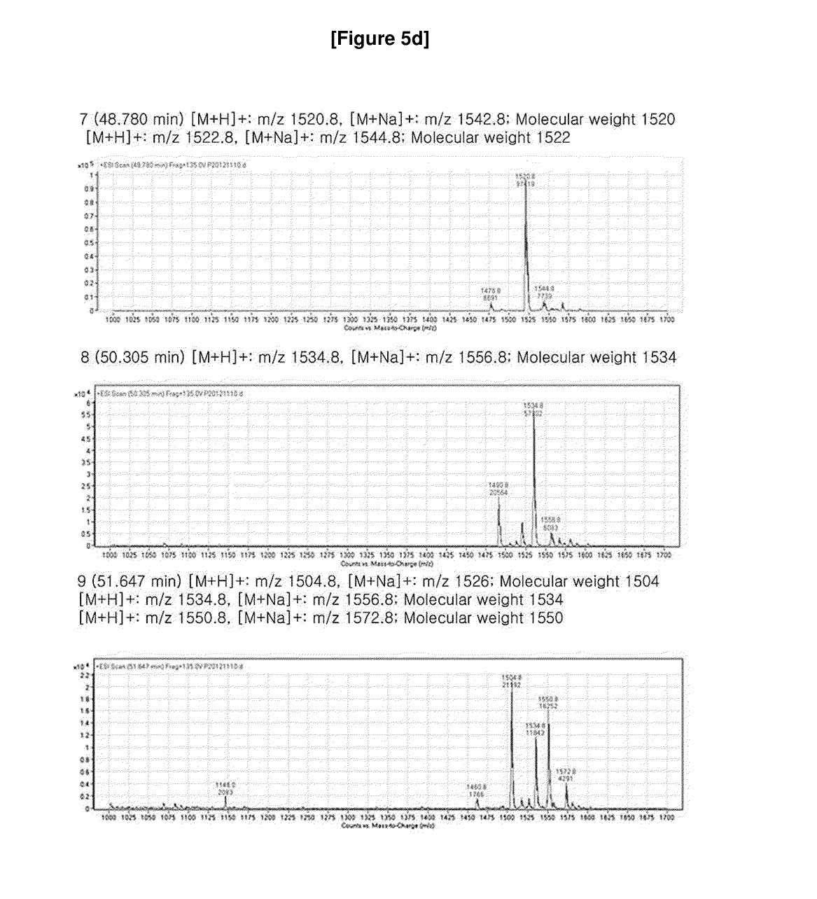

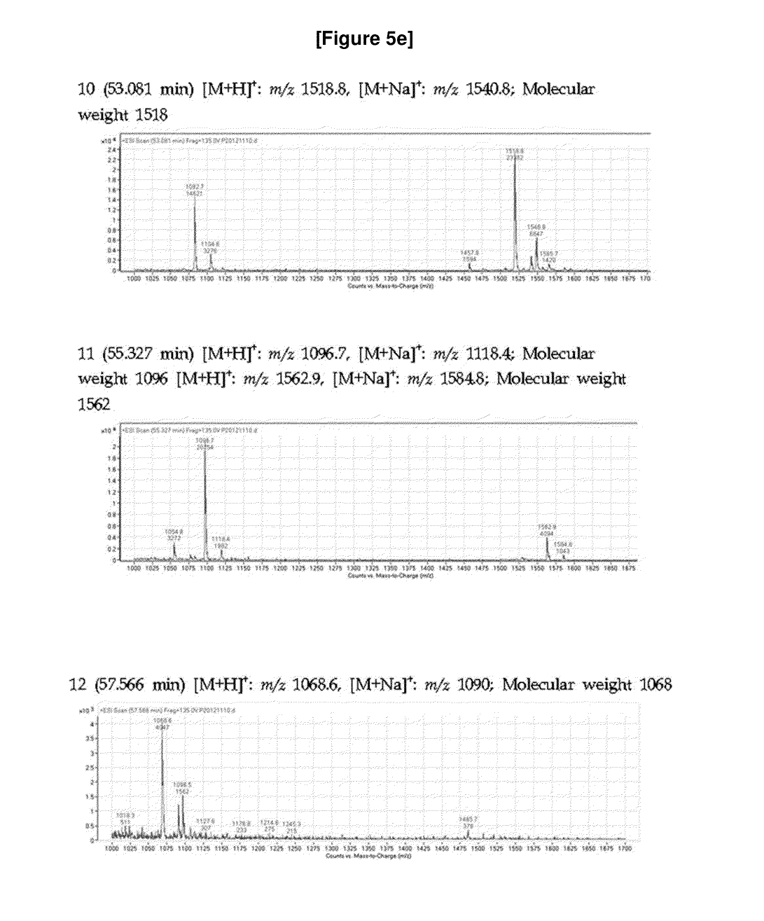

In the meantime, the region demonstrating HPLC retention time (37˜45 minutes) similar to that of the lipopeptide antibiotic fengycin was analyzed by LC-mass. As a result, the strain culture medium contained iturin and fengycin groups known as Bacillus sp. antibacterial materials (see FIG. 5).

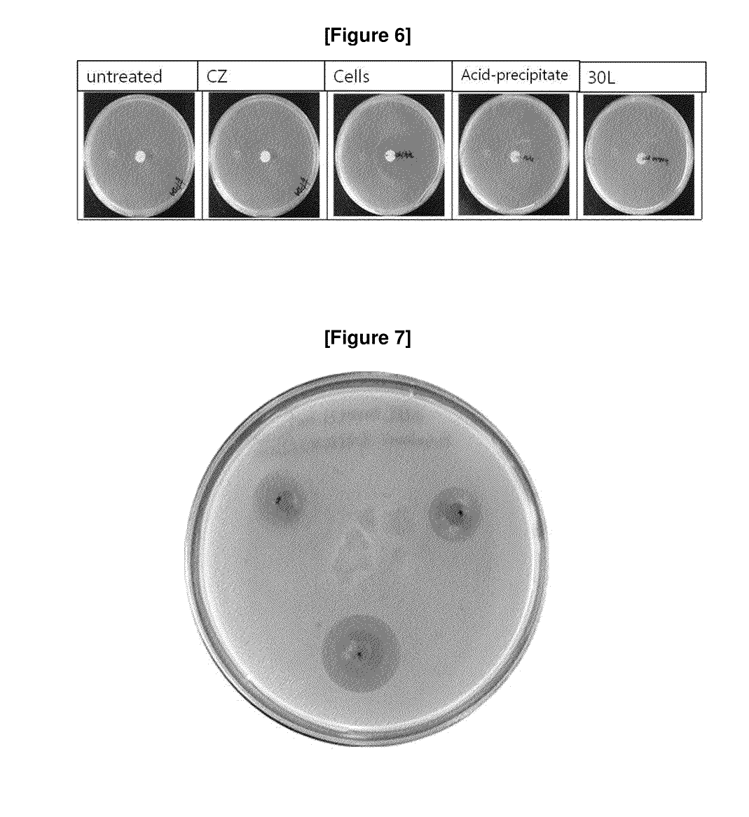

The antagonism of the novel Bacillus amyloliquefaciens KBC1004 strain of the present invention against the pathogen Rhizoctonia solani AG-2-2(IV) was investigated. As a result, it was confirmed that the novel Bacillus amyloliquefaciens KBC1004 strain contained the antibiotic material during the single liquid culture, suggesting that the antibiotic material was dormant in the inside of the cell, but once the strain received a stimulus like a pathogen, the gene involved in the delivery system and transport system in the novel Bacillus amyloliquefaciens KBC1004 strain was activated to secret the antibiotic material (see FIG. 6).

The growth inhibiting effect of the novel Bacillus amyloliquefaciens KBC1004 strain of the invention on plant pathogens was investigated. As a result, it was confirmed that the strain had excellent antifungal activity against various plant pathogens such as Rhizoctonia solani AG-2-2(IV) causing turfgrass diseases, Botrytis cinerea causing gray mold rot, Colletotrichum acutatum causing chili pepper anthracnose, Colletotrichum gleosprodes causing sweet persimmon anthracnose, Rhizoctonia cerealis causing yellow patch, Rhizoctonia solani AG-1(1A) causing brown patch, Sclerotium rolfsii causing southern blight, and Phytophthora drechsleri causing kiwifruit phytophthora blight. In particular, the antifungal activity of the strain against Rhizoctonia solani AG-2-2(IV) causing turfgrass diseases, Rhizoctonia cerealis causing yellow patch, and Colletotrichum acutatum causing chili pepper anthracnose was more significant (see Table 5).

The functional mechanism of the Bacillus amyloliquefaciens KBC1004 strain of the invention against plant pathogens including Rhizoctonia solani AG-2-2(IV) was investigated. As a result, Rhizoctonia solani AG-2-2(IV) formed a sensitizing band as a reaction against the Bacillus amyloliquefaciens KBC1004 strain, which suggested the Bacillus amyloliquefaciens KBC1004 strain interacted with Rhizoctonia solani AG-2-2(IV) so that Rhizoctonia solani AG-2-2(IV) secreted a sensitizing material to inhibit the growth of the pathogen itself (see FIG. 7).

Therefore, it was confirmed that the Bacillus amyloliquefaciens KBC1004 strain of the invention displayed excellent antifungal activity against various plant pathogens such as Rhizoctonia solani AG-2-2(IV) causing turfgrass diseases, Botrytis cinerea causing gray mold rot, Colletotrichum acutatum causing chili pepper anthracnose, Colletotrichum gleosprodes causing sweet persimmon anthracnose, Rhizoctonia cerealis causing yellow patch, Rhizoctonia solani AG-1(1A) causing brown patch, Sclerotium rolfsii causing southern blight, and Phytophthora drechsleri causing kiwifruit phytophthora blight, so that the strain could be efficiently used as an eco-friendly composition for controlling plant pathogens.

The present invention also provides a technique, a method, and a composition for controlling plant pathogens comprising the Bacillus amyloliquefaciens KBC1004) strain, the culture medium thereof, or the sensitizing material secreted by the interaction with the plant pathogens as an active ingredient.

The said plant pathogen is preferably one or more pathogens selected from the group consisting of Rhizoctonia solani AG-2-2(IV), Botrytis cinerea, Colletotrichum acutatum, Colletotrichum gleosprodes, Rhizoctonia cerealis, Rhizoctonia solani AG-1(1A), Sclerotium rolfsii, and Phytophthora drechsleri, but not always limited thereto.

The said sensitizing material is preferably obtained by the following procedure: culturing a plant pathogen on the medium containing the strain of claim 1 or the culture medium thereof; and collecting the sensitizing material secreted by the interaction between the strain or its culture medium and the plant pathogen by using methanol, but not always limited thereto.

In a preferred embodiment of the present invention, it was confirmed that the Bacillus amyloliquefaciens KBC1004 strain of the invention had excellent antifungal activity against various plant pathogens such as Rhizoctonia solani AG-2-2(IV), Botrytis cinerea, Colletotrichum acutatum, Colletotrichum gleosprodes, Rhizoctonia cerealis, Rhizoctonia solani AG-1(1A), Sclerotium rolfsii, and Phytophthora drechsleri. In particular, the antifungal activity of the strain against Rhizoctonia solani AG-2-2(IV) causing turfgrass diseases, Rhizoctonia cerealis causing yellow patch, and Colletotrichum acutatum causing chili pepper anthracnose was more significant (see Table 4).

Therefore, since the Bacillus amyloliquefaciens KBC1004 strain or the culture medium thereof of the present invention displays excellent antifungal activity against various plant pathogens, it can be efficiently used for the eco-friendly technique, method, and composition for controlling plant pathogens.

The present invention further provides a technique, a method, and a composition for controlling turfgrass rhizoctonia blight (large patch) caused by Rhizoctonia solani AG-2-2(IV) comprising the Bacillus amyloliquefaciens KBC1004 strain deposited under the Accession No. of KCTC 12355BP and the culture medium thereof as an active ingredient.

In a preferred embodiment of the present invention, the inventors prepared a liquid or a powder preparation using the Bacillus amyloliquefaciens KBC1004 strain of the present invention, which was then diluted in water at the ratio of 1:200˜1:1000. Soil drench was performed with the diluted preparation at the concentration of 1 L/m². One month after the soil drench, the damaged area by turfgrass rhizoctonia blight (large patch) was investigated to observe the controlling effect of the strain. As a result, the powder preparation of the Bacillus amyloliquefaciens KBC1004 strain displayed 90.1% controlling effect, suggesting that the strain is very practical as a natural plant protector (see Table 6).

Therefore, since the Bacillus amyloliquefaciens KBC1004 strain or the culture medium thereof of the present invention displays excellent antifungal activity against Rhizoctonia solani AG-2-2(IV) causing turfgrass rhizoctonia blight (large patch), it can be efficiently used as an eco-friendly composition for controlling rhizoctonia blight (large patch).

The present invention also provides a method for preparing a composition for controlling plant pathogens comprising the following steps:

1) preparing a culture product by culturing the Bacillus amyloliquefaciens KBC1004 strain deposited under the Accession No. of KCTC 12355BP in a liquid medium; and

2) preparing a raw material by drying the culture product prepared in step 1) and pulverizing thereof.

The medium of step 1) preferably contains dextrose at the concentration of 0.1˜10.0 weight part and peptone at the concentration of 0.1˜10.0 weight part, but not always limited thereto.

The culture in step 1) is preferably performed at 25˜35° C. under pH 5.5˜8.5, but not always limited thereto.

The composition of the present invention can be formulated in a suitable form for controlling plant pathogens, for example in the form of dried powders or liquid formulation, by the conventional method well known to those in the art.

Particularly, the composition can be formulated in the form of liquid bio-pesticides. To prepare the bio-pesticides as powders or granules, a diluent can be added to the bio-pesticides. However, the formulation is not limited thereto.

The composition can additionally contain a diluent, and at this time, the diluent plays a role in regulating the amount of the bio-pesticide composition in the total composition containing a microorganism or other active ingredients. A diluent usable in this invention is exemplified by sodium alginate, gelatinized starch, corn starch, soybean cake, wheat bran, granular fiber, ammonium sulfate, diatomite, zeolite, bentonite, talc, kaolin, pyrophyllite, white carbon, and saccharides.

The present invention also provides a method for controlling plant pathogens containing the step of treating an effective dose of the said strain, the culture medium thereof, or the sensitizing material secreted by the interaction with the plant pathogens to plants or cultivated soil.

The method for controlling plant pathogens using the composition of the invention can be achieved by the conventional methods, such as spraying (for example, nebulization, misting, atomizing, powder dispersing, granule dispersing, water surface application, phase application, etc), soil application (for example, mixing, drenching, etc), surface application (for example, spreading, smearing, coating, etc), soaking, smoking, etc. The dose of the composition can be determined by considering damage condition, application method, and application place, etc.

In a preferred embodiment of the present invention, the inventors prepared a liquid or a powder preparation using the Bacillus amyloliquefaciens KBC1004 strain of the present invention, which was then diluted in water at the ratio of 1:200˜1:1000. Soil drench was performed with the diluted preparation at the concentration of 1 L/m². One month after the soil drench, the damaged area by turfgrass rhizoctonia blight (large patch) was investigated to observe the controlling effect of the strain. As a result, the powder preparation of the Bacillus amyloliquefaciens KBC1004 strain displayed 90.1% controlling effect, suggesting that the strain is very practical as a natural plant protector (see Table 6).

Therefore, the method for controlling plant pathogens of the present invention is a promising eco-friendly method for controlling plant pathogens.

The preset invention also provides a use of the novel Bacillus amyloliquefaciens KBC1004 strain deposited under the Accession No. of KCTC 12355BP, the culture medium thereof, or the sensitizing material secreted by the interaction with the plant pathogens for the preparation of a composition for controlling plant pathogens.

Since the strain or the culture medium thereof of the present invention displays excellent antifungal activity against various plant pathogens, it can be efficiently used as an eco-friendly composition for controlling plant pathogens.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Isolation and Identification of Bacillus amyloliquefaciens KBC1004 Strain

A strain demonstrating excellent antibacterial activity was selected from the microorganisms collected from soil.

For the identification of the selected strain, the inventors asked Changwon University, Korea to analyze 16s rDNA sequence of the strain. As a result, the nucleotide sequence of FIG. 1 (SEQ. ID. NO: 1) was confirmed. Then, the inventors performed homology search by using NCBI GenBank database and confirmed its phylogenetic position. From the molecular phylogenetic analysis, the selected strain was identified as Bacillus amyloliquefaciens belonging to the Bacillus sp. phylogenetic group.

The isolated and identified strain was named Bacillus amyloliquefaciens KBC1004 and deposited at Korean Collection for Type Cultures (KCTC), Korea Research Institute of Bioscience and Biotechnology (KRIBB) on Jan. 18, 2013 (Accession No: KCTC 12355BP).

### Example 2

### Sequencing and Analyzing Bacillus amyloliquefaciens KBC1004 Strain Genome

Sequencing and analyzing the Bacillus amyloliquefaciens KBC1004 strain genome were performed at Korea Research Institute of Bioscience and Biotechnology (KRIBB).

Particularly, 2×100 paired end sequencing was performed with the Bacillus amyloliquefaciens KBC1004 strain by using Hiseq2000 (Illumina). As a result, approximately 500 bp fragment sequence was obtained, from which 30,257,430 sequences (3.06 Gb) were readable. After trimming the sequence using CLC Genomics Workbench Ver. 5.5, the readable sequence in the average length of 93.9 bp (total 2.48 Gb) was prepared. The sequence of 9.909,052 bp was identified by De novo assembly and reference mapping. As a reference sequence, Bacillus amyloliquefaciens FZB42 (3,918,589 bp) was used for mapping.

The paired read was aligned to the scaffold structure resulted from De novo assembly to fill up the gap. The sequence that filled partially the gap was handed in RAST server (http://rast.nmprd.org), followed by automatic genome annotation. The average nucleotide identity (ANI) of the common genes between the two strains can be used as a useful tool for measuring the genetic relation between the two strains. This method is not only simple but also applied to every kind of species and able to identify a strain at the level under species. 95˜96% ANI is the traditional 60˜70% DNA-DNA hybridization which is used as a standard for the identification of species. ANI analysis was performed by using JSpecies program. As a result, the strain of the invention showed 93.8% ANI with the type species Bacillus amyloliquefaciens subsp. amyloliquefaciens ATCC 23350T=DSM 7T, and particularly displayed 97.6% ANI with Bacillus amyloliquefaciens FZB42 (FIG. 2). Therefore, the Bacillus amyloliquefaciens KBC1004 strain of the invention was identified as Bacillus amyloliquefaciens.

RAST (Rapid Annotation Using Subsystem Technology) server was used for the genome annotation. The server provides an authentic automatic annotation of bacteria and Archaea genomes. Subsystem is an aggregate of functional roles composing metabolic pathway or protein complex or protein class. It is curated from multiple genomes by the expert annotator and then composes the protein family FlGfam, which is an important base for automatic annotation. At this time, the annotated genetic information is preserved in SEED framework. SEED indicates the annotation environment facilitating the project to annotation 1000 genomes.

Taking Bacillus amyloliquefaciens FZB42, the closest species to Bacillus amyloliquefaciens KBC1004, as a reference sequence, the antibiotic biosynthesis gene group was investigated.

As a result, as shown in Table 1, the Bacillus amyloliquefaciens KBC1004 of the invention was confirmed to have the genome involved in the biosynthesis of surfactin, bacillomycin D like antibiotic material, fengycin, putative peptide, bacillibactin, bacilysin/anticapsin, macrolactin, bacillaene, and difficidin (Table 1).

TABLE 1
Comparison of antibiotic biosynthesis gene group
between Bacillus amyloliquefaciens KBC1004 and
Bacillus amyloliquefaciens FZB42

| Antibiotic | Bacillus | Bacillus |
|---|---|---|
| biosynthesis gene | amyloliquefaciens | amyloliquefaciens |
| group | KBC1004 | FZB42 |
| Surfactin | ∘ | ∘ |
| Bacillomycin D | Bacillomycin D like | ∘ |
| | gene cluster | |
| Fengycin | ∘ | ∘ |
| Putative peptide | ∘ | ∘ |
| Bacillibactin | ∘ | ∘ |
| Bacilysin/anticapsin | ∘ | ∘ |
| Macrolactin | ∘ | ∘ |
| Bacillaene | ∘ | ∘ |
| Difficidin | ∘ | ∘ |

### Example 3

### Biochemical Characteristics of Bacillus amyloliquefaciens KBC1004 Strain

<3-1> Confirmation of the Enzyme Produced by Bacillus amyloliquefaciens KBC1004 Strain

The enzyme produced by the Bacillus amyloliquefaciens KBC1004 strain separated and identified in Example 1 was investigated.

Particularly, the enzyme productivity of the strain targeting 19 enzymes was investigated by using APi ZYM kit (BioMeriux, Lyon, France). According to the color changes, the level was defined from 0 to 5, precisely the negative reaction(−) is presented as 0 and when the obtained value is 3 or up it is judged as positive reaction(+).

As a result, as shown in Table 2, it was confirmed that the Bacillus amyloliquefaciens KBC1004 strain produced Esterase (C4) and Naphtol-AS-BI-phosphohydrolase (Table 2).

TABLE 2
Enzyme activity of Bacillus amyloliquefaciens KBC1004 strain
API 20 ZYM

| Enzymes detectables | Result | Enzymes detectables | Result |
|---|---|---|---|
| Control (Temoin sans substrate) | −¹⁾ | Acid phospatase (Phosphatases) | − |
| Alkaline phosphatase | − | Naphtol-AS-BI-phosphohydrolase | + |
| Esterase (C4) (Lipases) | + | α-galactosidase(melibiase) (Oxidases) | − |
| Esterase Lipase (C8) | − | β-galactosidase(lactase) | − |
| Lipase (C14) | − | β-Glucuronidase(hyaluronidase) | − |
| Leucine arylamidase (Proteases) | − | α-glucosidase(maltase) | − |
| Valine arylamidase | − | β-glucosidase(cellulase) | − |
| Crystine arylamidase | − | N-acetyl-β- | − |
| Trypsin | − | glucosaminidase(chitinase) | |
| α-chymotrypsin | − | α-mannosidase | − |
| | | α-fucosidase | − |

¹⁾+: positive, −: negative

<3-2> Carbohydrate Usability of Bacillus amyloliquefaciens KBC1004 Strain

To investigate carbohydrate usability of the Bacillus amyloliquefaciens KBC1004 strain, the oxidation, fermentation, and anabolism of 49 carbohydrates were investigated by using APl 50 CHB kit. When an acid was produced, the medium turned to yellow by the indicator phenol red included therein, which indicated the positive reaction. In the meantime, if the medium would turn red, it indicated the negative reaction. Also, in the esculin test, if the medium turned from red to black, it indicated the positive reaction. The results are shown in Table 3 (Table 3).

TABLE 3
Carbohydrate usability of Bacillus amyloliquefaciens KBC1004 strain
API 50 CHB

| | | | |
|---|---|---|---|
| Control | −¹⁾ | Esculin | + |
| Glycerol | − | Salicine | + |
| Erythritol | − | D-Cellobiose | + |
| D-Arabinose | − | D-Maltose | + |
| L-Arabinose | + | D-Lactose | + |
| D-Ribose | + | D-Melibiose | − |
| D-Xylose | + | D-Saccharose | + |
| L-Xylose | − | D-Trehalose | + |
| D-Adonitol | − | Inulin | − |
| Methyl-β-D-Xylopyranoside | − | D-Melezitose | − |
| D-Galactose | − | D-Raffinose | + |
| D-Glucose | + | Amidon (Starch) | + |
| D-Fructose | + | Glycogene | + |
| D-Mannose | + | Xylitol | − |
| L-Sorbose | − | Gentiobiose | + |
| L-Rhamnose | − | D-Turanose | − |
| Dulcitol | − | D-Lyxose | − |
| Inositol | − | D-Tagatose | − |
| D-Mannitol | + | D-Frucose | − |
| D-Sorbitol | + | L-Frucose | − |
| Methyl-α,D-Mannopyranoside | − | D-Arabitol | − |
| Methyl-α,D-Glucopyranoside | + | L-Arabitol | − |
| N-Acethyl-Glucosamine | + | Gluconate | − |
| Amygdalin | + | 2-keto-Gluconate | − |
| Arbutin | + | 5-keto-Gluconate | − |

¹⁾+: positive, −: negative

### Example 4

### Analysis of Antibacterial Material of Bacillus amyloliquefaciens KBC1004 Strain Working Against Plant Pathogens

To analyze the antibiotic material produced by the Bacillus amyloliquefaciens KBC1004 strain of the present invention, HPLC and LC-MS with the acid precipitate of the culture medium thereof was conducted by NPChem, Korea.

Particularly, pH of the culture medium (2 L) was regulated as 2 by adding HCl and then the sample stood at room temperature for 5 hours. The culture medium was centrifuged at 10,000 rpm for 30 minutes and then the supernatant was discarded. The precipitate was dissolved in methanol. The undissolved precipitate was eliminated by centrifugation and only the precipitate dissolved in methanol was concentrated under reduced pressure. The concentrated sample was dissolved again in 300 μl of methanol, which was used as an assay sample.

HPLC was performed by using the gradient solvent presented in Table 4. The solvent A was 5% acetonitrile/0.04% TFA and the solvent B was acetonitrile. The column used herein was ODS column and the flow rate was set at 1 ml/min.

TABLE 4

| | Solvent A (%) | Solvent | Flow rate |
|---|---|---|---|
| Time (min) | (5% ACN/0.04% TFA) | B (%) | (ml/min) |
| 0.0 | 90 | 10 | 1 |
| 80.0 | 0 | 100 | 1 |
| 85.0 | 90 | 10 | 1 |
| 90.0 | 90 | 10 | 1 |

As a result of HPLC, as shown in FIG. 3 and FIG. 4, multiple peaks were detected in the retention time of 37˜45 minutes. Along with the analysis of the retention time of the peak, UV spectrum presenting the maximum absorbance at 222 and 275 nm also suggested that the antibiotic material produced by the strain was fengycin cluster, the lipopeptide antibiotic (FIGS. 3 and 4).

In the meantime, the region demonstrating HPLC retention time (37˜45 minutes) similar to that of the lipopeptide antibiotic fengycin was analyzed by LC-mass.

As a result, as shown in FIG. 5, 12 TIC peaks were detected. The molecular weight of each compound was measured. As a result, [M+H]⁺ of compound 1 was observed at m/z 1538.8 and [M+Na]⁺ was observed at m/z 1560.8, indicating the molecular weight of compound 1 was 1538. Compound 2 was a mixture of 2 major compounds. [M+H]⁺ of the first compound was observed at m/z 1552.8 and [M+Na]⁺ was observed at m/z 1575.8, indicating the molecular weight of the compound was 1552. [M+H]⁺ of the second compound was observed at m/z 1566.8 and [M+Na]⁺ was observed at m/z 1588.8, indicating the molecular weight of the compound was 1566. [M+H]⁺ of compound 3 was observed at m/z 1523.8 and [M+Na]⁺ was observed at m/z 1545.8, indicating the molecular weight of compound 3 was 1523. [M+H]⁺ of compound 4 was observed at m/z 1538.8 and [M+Na]⁺ was observed at m/z 1560.8, indicating the molecular weight of compound 4 was 1538. Compound 5 was a mixture of 2 major compounds. [M+H]⁺ of the first compound was observed at m/z 1506.8 and [M+Na]⁺ was observed at m/z 1528.7, indicating the molecular weight of the compound was 1506. [M+H]⁺ of the second compound was observed at m/z 1552.8 and [M+Na]⁺ was observed at m/z 1574.8, indicating the molecular weight of the compound was 1552. [M+H]⁺ of compound 6 was observed at m/z 1566.9 and [M+Na]⁺ was observed at m/z 1588.8, indicating the molecular weight of compound 6 was 1566. Compound 7 was a mixture of 2 major compounds. [M+H]⁺ of the first compound was observed at m/z 1520.8 and [M+Na]⁺ was observed at m/z 1542.8, indicating the molecular weight of the compound was 1520. [M+H]⁺ of the second compound was observed at m/z 1522.8 and [M+Na]⁺ was observed at m/z 1544.8, indicating the molecular weight of the compound was 1522. [M+H]⁺ of compound 8 was observed at m/z 1534.8 and [M+Na]⁺ was observed at m/z 1556.8, indicating the molecular weight of compound 8 was 1534. Compound 9 was a mixture of 3 major compounds. [M+H]⁺ of the first compound was observed at m/z 1504.8 and [M+Na]⁺ was observed at m/z 1526, indicating the molecular weight of the compound was 1504. [M+H]⁺ of the second compound was observed at m/z 1534.8 and [M+Na]⁺ was observed at m/z 1556.8, indicating the molecular weight of the compound was 1534. [M+H]⁺ of the third compound was observed at m/z 1550.8 and [M+Na]⁺ was observed at m/z 1572.8, indicating the molecular weight of the compound was 1550. [M+H]⁺ of compound 10 was observed at m/z 1518.8 and [M+Na]⁺ was observed at m/z 1540.8, indicating the molecular weight of compound 10 was 1518. Compound 11 was a mixture of 2 major compounds. [M+H]⁺ of the first compound was observed at m/z 1096.7 and [M+Na]⁺ was observed at m/z 1118.4, indicating the molecular weight of the compound was 1096. [M+H]⁺ of the second compound was observed at m/z 1562.9 and [M+Na]⁺ was observed at m/z 1584.8, indicating the molecular weight of the compound was 1562. [M+H]⁺ of compound 12 was observed at m/z 1068.6 and [M+Na]⁺ was observed at m/z 1090, indicating the molecular weight of compound 12 was 1068.

Based on the obtained molecular weight data, database and literature search was performed. As a result, it was confirmed that the compound displayed fengycin like HPLC retention time and had a little bigger molecular weight than that of fengycin. Compound 11 and compound 12 had the molecular weight similar to that of the iturin compound.

Therefore, the compounds were confirmed to be Iturin and fengycin groups, known as Bacillus sp. antibacterial materials, (FIG. 5).

### Example 5

### Activation of Antibacterial Material Working Against Plant Pathogen by Bacillus amyloliquefaciens KBC1004 Strain

To investigate the antagonism of the Bacillus amyloliquefaciens KBC1004 strain against Rhizoctonia solani AG-2-2(IV), Rhizoctonia solani AG-2-2(IV) was placed on the sterilized potato agar (diluted 1/5) medium, to which 10 μl of the Bacillus amyloliquefaciens KBC1004 culture fluid which had been cultured for 24 hours was inoculated, followed by replacement culture at 30° C. for 5 days. Inhibition zone formed between the two strains was taken off and extracted with methanol. The extract was concentrated and named “CZ”.

The Bacillus amyloliquefaciens KBC1004 strain of the invention was shaking-cultured in LB medium at 30° C. at 120 rpm for 3 days, resulting in the preparation of the first inoculum. The prepared first inoculum was inoculated in 30 L of LB medium, followed by shaking-culture at 30° C. at 120 rpm for 3 days. The cells and the culture medium were separated from each other by ultracentrifugation. The obtained supernatant was concentrated to 1/10, which was then named “30 L”.

The remaining cells were extracted with ethanol and the extract was named “cells”

Acid precipitation was performed with the 30 L culture medium supernatant by using HCl. And the resultant acid precipitate was named “acid precipitate”. Then, the potato agar medium (1/5 diluted) was added with Rhizoctonia solani AG-2-2(IV), CZ, 30 L, cells, and acid precipitate, followed by culture at 30° C. for 3 days.

As a result, as shown in FIG. 6, the CZ and cell extract displayed the antagonistic effect on pathogen, but the culture medium concentrate (30 L) and acid precipitate did not display the antagonistic effect on pathogen (FIG. 6). Therefore, it was confirmed that the Bacillus amyloliquefaciens KBC1004 strain of the present invention contained the antibiotic material during the single liquid culture, suggesting that the antibiotic material was dormant in the inside of the cell, but once the strain received a stimulus like a pathogen, the gene involved in the delivery system and transport system in the Bacillus amyloliquefaciens KBC1004 strain was activated to secret the antibiotic material.

### Example 6

### Antibacterial Activity of Bacillus amyloliquefaciens KBC1004 Strain Against Plant Pathogens

To investigate whether or not the Bacillus amyloliquefaciens KBC1004 strain separated and identified in Example 1 and deposited under the Accession No. of KCTC 12355BP had the growth inhibition effect on plant pathogens, the Bacillus amyloliquefaciens KBC1004 strain was cultured with various plant pathogens on the sterilized potato agar medium. At this time, as the plant pathogens, each strain that had been sub-cultured in the potato agar medium was sliced into a round section of 5 mm in diameter. The Bacillus amyloliquefaciens KBC1004 cultured in LB medium at 28° C. for 24 hours was distributed in 5 mm paper disk (20 μl), followed by replacement culture with the prepared slices. After culturing the sample for 10 days at 28° C. which is the well growing temperature for various pathogens, the diameter of the inhibition zone was measured.

As the plant pathogens, Rhizoctonia solani AG-2-2(IV), Botrytis cinerea, Colletotrichum acutatum, Colletotrichum gleosprodes, Rhizoctonia cerealis, Rhizoctonia solani AG-1(1A), Sclerotium rolfsii, and Phytophthora drechsleri were used.

As a result, as shown in Table 5, it was confirmed that the Bacillus amyloliquefaciens KBC1004 strain of the present invention had excellent antifungal activity against all the plant pathogens used herein, and particularly the antifungal activity against Rhizoctonia solani AG-2-2(IV) causing turfgrass diseases, Rhizoctonia cerealis causing yellow patch, and Colletotrichum acutatum causing chili pepper anthracnose was more significant (Table 5).

TABLE 5
Antifungal spectrum of Bacillus amyloliquefaciens
KBC1004 strain

| | Antifungal |
|---|---|
| Plant pathogen | activity |
| Botrytis cinerea KACC40574 | ++++ |
| Botrytis cinerea KACC43521 | +++ |
| Colletotrichum acutatum | +++++ |
| Sclerotium rolfsii | ++ |
| Rhizoctonia cerealis | +++++ |
| Rhizoctonia solani AG-1(1A) | ++++ |
| Rhizoctonia solani AG-2-2(IV) | +++++ |
| Colletotrichum gleosprodes | ++++ |
| Phytophthora drechsleri | ++ |

+: 0 < X 10;
++: 10 < X 20;
+++: 20 < X 30;
++++: 30 < X 40; and
+++++: 40 < X.

### Example 7

### Functional Mechanism of Bacillus amyloliquefaciens KBC1004 Strain Against Plant Pathogens Including Rhizoctonia solani AG-2-2(IV) and Establishment of Method for Collecting Sensitizing Material

To investigate the functional mechanism of the Bacillus amyloliquefaciens KBC1004 strain against plant pathogens including Rhizoctonia solani AG-2-2(IV), 3 ml of Bacillus amyloliquefaciens KBC1004 solution was inoculated in 300 ml of 1.5% potato agar medium (diluted 1/5), resulting in the bacteria solution medium. The prepared bacteria solution medium was distributed in petridish. Once the medium was hardened, the medium was punched with cork ball #8. 1.5% agar was filled in the hole, which was hardened. Rhizoctonia solani AG-2-2(IV) in the round slice of 5 mm in diameter was placed thereon. 24 hours later, the medium was observed by the naked eye.

As a result, as shown in FIG. 7, Rhizoctonia solani AG-2-2(IV) formed a sensitizing band, indicating that the pathogen reacted to the Bacillus amyloliquefaciens KBC1004 strain. The sensitizing material secreting area was cut off and extracted with methanol. The extract was concentrated, and the concentrate was replacing-cultured with Rhizoctonia solani AG-2-2(IV). As a result, it was confirmed that Rhizoctonia solani AG-2-2(IV) formed the inhibition zone to the sensitizing material (FIGS. 6 and 7). That is, the Bacillus amyloliquefaciens KBC1004 strain of the invention interacted with Rhizoctonia solani AG-2-2(IV) and accordingly Rhizoctonia solani AG-2-2(IV) secreted a sensitizing material, resulting in the inhibition of the growth of the pathogen itself. Therefore, the method for collecting the material was established.

### Example 8

### Formulation of Bacillus amyloliquefaciens KBC1004 Strain

The plant pathogen control agent comprising the Bacillus amyloliquefaciens KBC1004 strain of the invention was prepared in the forms of liquid and power.

Particularly, the strain was inoculated in a liquid medium supplemented with dextrose (1%, w/w) and peptone (1%, w/w), followed by shaking-culture at 28° C. under pH 7.0 for 3 days. Then, the culture fluid was used as a raw material for the liquid preparation formula and the powder preparation.

To prepare the liquid preparation, a stabilizer such as potassium solvate was added to the liquid raw material at the concentration of 0.1˜0.5%. To prepare the powder preparation, the Bacillus amyloliquefaciens KBC1004 strain raw material (5.0˜50.0%, w/w) was mixed with a diluent (50.0˜95.5%, w/w), followed by spray-drying.

### Example 9

### Controlling Effect of Bacillus amyloliquefaciens KBC1004 on Plant Pathogens

In the early stage of turfgrass rhizoctonia blight (large patch) developed on the grass (Zoysiagrass) in the area of Sacheon-si, Gyeongsangnam-do, Korea, the powder preparation prepared in Example 5, 200˜1000 times diluted in water, was drenched on the soil of that area once at the concentration of 1 L/m² (In May 25, 2011). One month after the soil drench, the damaged area by turfgrass rhizoctonia blight (large patch) was investigated to observe the controlling effect of the strain.

As a result, the powder preparation of the Bacillus amyloliquefaciens KBC1004 strain displayed 90.1% controlling effect, suggesting that the strain is very practical as a natural plant protector (Table 6).

TABLE 6
Controlling effect of Bacillus amyloliquefaciens
KBC1004 powder preparation on turfgrass rhizoctonia
blight (large patch)

| | Treatment | Damaged | Controlling |
|---|---|---|---|
| Sample | Method | area (%) | effect (%) |
| 200 x | Soil Drench | 3.18 | 90.1 |
| 500 x | | 3.51 | 89.1 |
| 1000 x | | 5.14 | 84.0 |
| Non-treated | | 32.12 | — |

### INDUSTRIAL APPLICABILITY

The composition for controlling plant pathogens comprising the Bacillus amyloliquefaciens KBC1004 strain of the invention or the culture medium thereof as an active ingredient displays excellent antifungal activity against various plant pathogens, so that it can be efficiently used for the preparation of an eco-friendly composition for controlling plant pathogens. Therefore, the concerned problems such as the destruction of ecosystem and toxicity on human/animal caused by synthetic agricultural chemicals can be overcome by using the above.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A novel Bacillus amyloliquefaciens KBC1004 strain deposited under the Accession No. of KCTC 12355BP.

2. The novel Bacillus amyloliquefaciens KBC1004 strain according to claim 1, wherein the strain has the antibacterial activity against one or more plant pathogens selected from the group consisting of Rhizoctonia solani AG-2-2(IV), Botrytis cinerea, Colletotrichum acutatum, Colletotrichum gleosprodes, Rhizoctonia cerealis, Rhizoctonia solani AG-1(1A), Sclerotium rolfsii, and Phytophthora drechsleri.

3. A composition for controlling plant pathogens comprising the strain of claim 1, the culture medium of the same, or the sensitizing material secreted by the interaction with the plant pathogens as an active ingredient.

4. The composition for controlling plant pathogens according to claim 3, wherein the sensitizing material is collected by the following procedure: culturing a plant pathogen on the medium containing the strain of claim 1 or the culture medium thereof; and collecting the sensitizing material secreted by the interaction between the strain or its culture medium and the plant pathogen by using methanol.

5. A method for controlling plant pathogens containing the step of treating an effective dose of the strain of claim 1, the culture medium of the same, or the sensitizing material secreted by the interaction with the plant pathogens to plants or cultivated soil.

6. A use of the novel Bacillus amyloliquefaciens KBC1004 strain deposited under the Accession No. of KCTC 12355BP, the culture medium thereof, or the sensitizing material secreted by the interaction with the plant pathogens for the preparation of a composition for controlling plant pathogens.


SEQUENCE LISTING (1 sequence)

SEQ ID NO: 1 - DNA, 708 bp, Bacillus amyloliquefaciens
  misc_feature (141)..(141): n is a, c, g, or t
gtgcctaatacatgcaagtcgagcggacagatgggagcttgctccctgatgttagcggcggacgggtgagtaacacgtgggtaacctgcctgtaagactgggataactccgggaaaccggggctaataccggatggttgtntgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggaggggtcattgggaaactggggaacttgagtgcagaagaggagagtggaattccacgtgttaccggtgaaatgcgtaaaaatgtggangaaccccagtggcgaangcgactctctg